(19) United States
(12) Patent Application Publication
(10) Pub. No.: US 2022/0265739 A1
(43) Pub. Date: Aug. 25, 2022

(54) Bacteriophage Hydrogel Compositions and Uses Thereof

(71) Applicant: McMaster University, Hamilton (CA)

(72) Inventor: Zeinab Hosseinidoust, Dundas (CA)

(21) Appl. No.: 17/626,295

(22) PCT Filed: Jul. 13, 2020
(86) PCT No.: PCT/CA2020/050975

§ 371 Date: Jan. 11, 2022

Related U.S. Application Data
(60) Provisional application No. 62/872,904, filed on Jul. 11, 2019.

(51) Int. Cl. A61K 35/76; A61K 47/69; A61P 17/02; A61P 31/04; C12N 7/02; C12Q 1/70; C12N 15/09; A61K 47/10

(52) U.S. Cl. CPC A61K 35/76; A61K 47/6901; A61K 47/6903; A61P 17/02; A61P 31/04; C12N 7/02; C12Q 1/70; C12N 15/09; A61K 47/10; C12N 2795/14132; C12N 2795/14151

(57) ABSTRACT

Described herein are hydrogel compositions comprising cross-linked bacteriophages. The hydrogels are typically bioactive, degradable, for example biodegradable, self-healing, fluorescent, for example autofluorescent, and/or birefringent. The hydrogels described herein may be used as therapeutics or diagnostics, as scaffolds for material synthesis, as catalysts, as membranes or filters, or as biosensor substrates, for example.

### FIELD

The present disclosure relates to hydrogel compositions, and in particular, to hydrogel compositions comprising bacteriophages.

### BACKGROUND

Bacteriophages (i.e. bacterial viruses) are essentially bionanoparticles with a protein coat, the composition of which can be controlled with atomic precision via genetic engineering, a property that makes them superior to synthetic nanoparticles as building blocks for bottom-up synthesis of multifunctional materials with advanced properties. Filamentous bacteriophages (phage) of Escherichia coli (f1, M13 and fd) first garnered attention as platforms for peptide/protein display in phage display technology, then later for their ability to form nanomaterials, such as chiral nematic liquid crystals, as well as their ability to express peptides binding to non-organic ligands, such as metals and plastics.

Filamentous phage are semi-flexible proteinous nanoscale filaments with a very high aspect ratio (0.88 μm in length and 6.6 nm in diameter, with a molecular weight of 1.6×10⁷ g/mol for wild type M13). As one of the most widely investigated in this family, the M13 nanofilament is composed of 2700 copies of the major structural protein (protein 8) along its length and is capped on each end with two different proteins. This protein shell encases a circular, single stranded DNA that encodes 11 genes, 5 of which code the 5 structural proteins of the virion. The remaining 6 genes are required to help the virus infect its host bacterial cell and turn the bacterial cell into a factory for making thousands of M13 nanofilaments. Genetic engineering to control the chemistry of the bacteriophage protein coat (all 5 structural proteins) allows for the expression of multiple different peptides/proteins/antibodies on the M13 protein coat, each offering specific interaction/recognition with a different ligand. Combined with the ability of these nanofilaments to make thousands of identical copies of themselves under mild physiological conditions and at room temperature (simply by infecting a culture of host bacteria), and the ability to encode for new forms of biorecognition via the powerful phage display technology, filamentous phage in general and M13 in particular claim a unique spot in a biological engineer's toolbox.

As such, work on the use of filamentous phage as a tool for material design have focused on the ability of M13 to be screened (through phage display) for peptides that bind to proteins, enzymes, cells, or minerals, hence providing a substrate with tunable biorecognition.

### SUMMARY

In accordance with an aspect, there is provided a hydrogel composition comprising cross-linked bacteriophages.

In an aspect, the bacteriophages self-assemble into bundles.

In an aspect, the bacteriophages comprise filamentous bacteriophages.

In an aspect, the bacteriophages comprise Escherichia coli bacteriophages.

In an aspect, the bacteriophages comprise f1, M13, or fd bacteriophages, or combinations thereof.

In an aspect, the bacteriophages comprise M13 bacteriophages.

In an aspect, the bacteriophages comprise covalent crosslinks.

In an aspect, the bacteriophages comprise non-covalent crosslinks.

In an aspect, the bacteriophages are crosslinked with one or more crosslinkers comprising polyelectrolytes, nanoparticles and/or nanocrystals.

In an aspect, the one or more crosslinkers comprises glutaraldehyde.

In an aspect, the hydrogel exhibits one or more of the following properties:
bioactivity, for example antibacterial activity;
degradability, such as biodegradability;
self-healing, optionally in the presence of calcium ions and/or phosphate-buffered saline;
fluorescent, such as autofluorescent; and/or
birefringence.

In an aspect, the hydrogel comprises at least about 10⁸ PFU/mL bacteriophage, such as at least about 10⁹ PFU/mL, about 10¹⁰ PFU/mL, about 10¹¹ PFU/mL, about 10¹² PFU/mL, about 10¹³ PFU/mL, about 10¹⁴ PFU/mL, about 10¹⁵ PFU/mL, or about 10¹⁶ PFU/mL, such as from about 10⁸ PFU/mL to about 10¹⁶ PFU/mL of bacteriophage.

In an aspect, the hydrogel is dried to form an aerogel or xerogel.

In an aspect, the hydrogel is dried to form an aerogel by critical-point drying or freeze-drying.

In an aspect, the hydrogel further comprises one or more molecules for cell targeting and/or infectivity.

In an aspect, the hydrogel comprises genetically engineered bacteriophages.

In an aspect, the bacteriophages are genetically engineered for selective target ligand recognition.

In an aspect, the length of the filamentous bacteriophages is further tuned through gene-modification, giving the phage-composed hydrogel structure colors.

In an aspect, the hydrogel comprises at least two different bacteriophage strains.

In an aspect, the at least two different bacteriophage strains target the same bacterial species or different bacterial species to treat complex infections.

In an aspect, the hydrogel further comprises at least one polymer.

In an aspect, the at least one polymer comprises PEG.

In an aspect, the hydrogel excludes a polymer, for example excluding PEG.

In an aspect, the hydrogel further comprises microstructures on the surface.

In an aspect, the hydrogel is for transferring to microgels.

In an aspect, the hydrogel further comprises a bioactive agent, optionally wherein the bioactive agent is encapsulated within the hydrogel.

In an aspect, the bioactive agent comprises an antibiotic, optionally wherein the bacteriophages and antibiotic are in synergistic amounts.

In accordance with an aspect, there is provided a therapeutic or diagnostic comprising the hydrogel composition described herein.

In an aspect, the therapeutic or diagnostic further comprises a pharmaceutically acceptable carrier, diluent and/or adjuvant.

In an aspect, the therapeutic or diagnostic comprises a wound dressing

In accordance with an aspect, there is provided a scaffold for material synthesis comprising one or more of the hydrogel compositions described herein.

In accordance with an aspect, there is provided a catalyst comprising one or more of the hydrogel compositions described herein.

In accordance with an aspect, there is provided a bioactive membrane or filter comprising one or more of the hydrogel compositions described herein.

In accordance with an aspect, there is provided a biosensor substrate comprising one or more of the hydrogel compositions described herein.

In accordance with an aspect, there is provided a method for making a hydrogel composition, the method comprising crosslinking a suspension of bacteriophages

In an aspect, the bacteriophages are in a water suspension.

In an aspect, the suspension comprises at least about 10⁸ PFU/mL bacteriophage, such as at least about 10⁹ PFU/mL, about 10¹⁰ PFU/mL, about 10¹¹ PFU/mL, about 10¹² PFU/mL, about 10¹³ PFU/mL, about 10¹⁴ PFU/mL, about 10¹⁵ PFU/mL, or about 10¹⁶ PFU/mL, such as from about 10⁸ PFU/mL to about 10¹⁶ PFU/mL of bacteriophage.

In an aspect, crosslinking comprises mixing the bacteriophages with a crosslinking agent.

In an aspect, the cross-linking agent comprises glutaraldehyde, optionally at from about 0.1% to about 10%, such as from about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, or about 9.5% to about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5% or about 10%.

In an aspect, mixing comprises incubating with the crosslinking agent for a period of time, such as from about 1 hour to about 48 hours, such as from about 12 hours to about 24 hours, at a temperature of from about 4° C. to about 37° C., such as about room temperature.

In accordance with an aspect, there is provided a hydrogel composition made by the methods described herein.

Other features and advantages will become apparent from the following detailed description. It should be understood, however, that the detailed description and the specific examples, while indicating aspects, are given by way of illustration only and the scope of the claims should not be limited by these aspects, but should be given the broadest interpretation consistent with the description as a whole.

### DRAWINGS

Certain aspects will now be described in greater detail with reference to the attached drawings in which:

FIG. 1 shows (A) a schematic representation of the process used to prepare high concentrations of pure M13 phage—the process starts by propagating M13 in an exponentially growing culture of host bacteria, which is then separated from phage via centrifugation and then the crude phage stock was purified through two steps of PEG/NaCl precipitation followed by ultrafiltration; (B) AFM image of a monolayer of M13 phage assembled on freshly cleaved mica; the lighter portions are g3p's protruding from the end of the phage filaments (scale bar represents 500 nm); (C) the final concentrated M13 suspension, imaged with a polarized light microscope (scale bar represents 100 μm); (D) the same phage suspension as part C, observed without polarizers (scale bar represents 100 μm) in exemplary aspects.

FIG. 2 shows electron micrographs of M13 hydrogels prepared with a phage concentration of 3×10¹⁴ (A,B,C) and 3×10¹³ PFU/mL (D,E,F); all hydrogels were critical-point dried except for (F), which was freeze-dried (arrows on panel C show M13 bundles) in exemplary aspects.

FIG. 3 shows (A) pictures of glutaraldehyde-crosslinked M13 gels made with different M13 concentrations (concentrations are presented in terms of PFU/mL; all gels shown were crosslinked with 1% glutaraldehyde); (B) rheology test for the lowest limit of M13 concentration to form a hydrogel compared to rheological behavior of a phage suspension with the same concentration and glycerol as reference; (C) compression modulus for M13 hydrogels shown in part B; (D) swelling ratio for phage hydrogels with different M13 concentrations (legend shows M13 concentration in PFU/mL+Glutaraldehyde (GA) concentration in weight percent and images of a freeze-dried M13 hydrogel before and after 5 hours of swelling in PBS in exemplary aspects.

FIG. 4 shows (A) stress-strain curve for M13 phage hydrogels; (B) swelling ratio for 3×10¹³ PFU/mL hydrogels at different temperatures in exemplary aspects.

FIG. 5 shows (A) UV-Vis spectra of M13 suspension and M13 hydrogel; (B) the FTIR spectra for M13 hydrogel, M13 suspension and glutaraldehyde—the peaks for amide I, II, and III are marked with broken lines on x-axis; (C) emission scan for M13 hydrogels when excited at λ=470 nm—emission scans show distinctive emission peaks for M13 hydrogels, different from the constituting components (M13, and glutaraldehyde, and water); (D) fluorescence micrographs for hydrogel made with 3×10¹⁴ PFU/mL of M13: I. brightfield, II. hydrogel excited at 358 nm and imaged using a λ=461 nm optical filter, III. hydrogel excited at 598 nm and imaged using a λ=615 nm optical filter, IV. hydrogel excited at 498 nm and imaged using a λ=509 nm optical filter; (E) M13 hydrogel digested by proteinase K—images show biodegradation with time up to 36 hours; (F) fluorescence micrographs for proteinase K degradation product of M13 hydrogel showing autofluorescence of degradation products; the gain was kept constant for all three excitation/emission pairs (scale bar (c, d)=200 μm) in exemplary aspects.

FIG. 6 shows (A) emission scans when excited at 2=598 nm, respectively, and fluorescence images of (B) 3×10¹³ PFU/mL M13 phage hydrogel, (C) 3×10¹⁴ PFU/mL M13 phage suspension, (D) 3×10¹³ PFU/mL M13 phage suspension, (E) glutaraldehyde (scale bar (B-E)=200 μm) in exemplary aspects.

FIG. 7 shows phage hydrogel before and after repetitive healing in a 2.4 mM CaCl₂ solution: I. M13 hydrogel made with 3×10¹³ PFU/mL of phage, cut II. hydrogels in I, self-healed hydrogel after 48 hrs of incubation, III. same self-healed hydrogel, marked by broken lines on the location of the old and new cut, IV. hydrogel in III, cut, V. self-healed hydrogel, after the second cut (both scars are visible in the image) in exemplary aspects.

FIG. 8 shows (A) phage hydrogels with two different M13 concentrations before and after repetitive healing in a 2.4 mM CaCl₂ solution: I. M13 hydrogel made with 3×10¹³ PFU/mL (left) of phage and one with 3×10¹⁴ PFU/mL (right), II. hydrogels in I, cut with a scalpel, III. self-healed hydrogel after 48 hrs of incubation (top) and same self-healed hydrogel, marked by broken line on the location of new cut (bottom), IV. same self-healed hydrogel, cut at a different location, V. self-healed after the second cut (both scars are visible in the image); (B) compression behavior for M13 hydrogel before and after self-healing; (C,D) SEM images of M13 hydrogel after self-healing (arrows show the location of the scar) in exemplary aspects.

FIG. 9 shows phage hydrogel before and after repetitive healing in a PBS, 2.4 mM CaCl₂, and 0.9% saline solution: I. M13 hydrogel made with 3×10¹³ PFU/mL of phage, cut II. hydrogels in I, III. hydrogel after 24-48 hrs in salt solution in exemplary aspects.

FIG. 10 shows fluorescence images of 3×10¹⁴ PFU/mL M13 phage hydrogel before and after self-healing in CaCl2 for 24 hrs (scale bar (B-E)=200 μm) in exemplary aspects.

FIG. 11 shows (A) bioactive property of M13 hydrogels demonstrated by the ability of M13 in the hydrogel to infect bacterial cells—the wash waters from the gels did not exhibit detectable levels of free M13; (B) proposed mechanism for the observed bioactivity of the M13 phage hydrogels towards a culture of the host bacteria using a schematic representation of the M13 phage filament, showing genome and the tail fibers (g3p) in exemplary aspects.

FIG. 12 shows birefringent properties of M13 bacteriophage-based hydrogels: (A) M13 bacteriophages suspended in solution a viral concentration of 5×10¹⁴ PFU/mL display characteristic self-assembly-birefringent properties when rotated 360° on a polarized light microscope; (B) bacteriophage-based hydrogel containing a viral concentration of 1×10¹⁴ PFU/mL complemented with a 1% glutaraldehyde solution acting as a crosslinking agent. The resulting phage-hydrogel observes clear evidence of birefringent properties when rotated 360° on a polarized light microscope; (C) bacteriophage-based hydrogel containing a viral concentration of 2×10¹⁴ PFU/mL complemented with a 1% glutaraldehyde solution acting as a crosslinking agent in exemplary aspects.

FIG. 13 shows (A) picture of 5×10¹³ PFU/mL M13 phage hydrogels inside syringes crosslinked by 1-ethyl-3-(3-dimethylaminopropyl) carbodiimide (EDC) at the concentration of 5% in I and 2% in II; (B) I. AFM image of hydrogel made with 5×10¹³ PFU/mL of M13 and 2% EDC; II. enlarged drawing of red box in image I; (C) fluorescence micrographs for hydrogel made with 5×10¹³ PFU/mL of M13 and 2% EDC: I. brightfield, II. hydrogel excited at 358 nm and imaged using a λ=461 nm optical filter, III. hydrogel excited at 598 nm and imaged using a k=615 nm optical filter, IV. hydrogel excited at 498 nm and imaged using a λ=509 nm optical filter; (scale bar (I-IV)=200 μm) in exemplary aspects.

FIG. 14 shows SEM images of (A) porous mold with spherical pores, (B) M13 phage hydrogel displaying microbumps on the surface and (C) hydrogel displaying microscale suction cups—inserts are picture of (A) porous mold and (B) phage hydrogel in exemplary aspects.

FIG. 15 shows SEM images of microgels in exemplary aspects.

### DETAILED DESCRIPTION

Described herein is a hydrogel composition comprising cross-linked bacteriophages. In aspects, described herein is the use of self-organized bacteriophages, such as filamentous M13, as building blocks for bottom-up synthesis to develop hierarchically-structured soft matter. Typically, these hierarchically-structured hydrogels of self-organized, crosslinked bacteriophage bundles are comprised of hundreds of phage nanofilaments that impart both long-range and micron-scale order. In typical aspects, the hydrogels can adsorb up to 16× their weight in water, such as at least about 1×, about 2×, about 3×, about 4×, about 5×, about 6×, about 7×, about 8×, about 9×, about 10×, about 11×, about 12×, about 13×, about 14×, about 15×, or about 16× their weight in water. In additional or alternative aspects, the hydrogel compositions described herein exhibit advanced properties at room temperature, such as self-healing under biological conditions, autofluorescence in three channels that decays through biodegradation, offering non-destructive imaging capability, and bioactivity in the crosslinked state towards host bacteria. The latter is a particularly powerful property, allowing the development of hydrogels with tunable bioactivity when combined with phage display and/or recombinant DNA technology. In particular aspects, the hydrogels are comprised of two components, namely filamentous bacteriophages and a crosslinker, such as glutaraldehyde. These two components provide the aforementioned properties without functionality from additional components or responsive crosslinkers. Thus, in aspects, additional polymers may be excluded from the compositions described herein. In other aspects, such polymers may be included in the compositions described herein.

Accordingly, described herein is a hydrogel composition comprising a physically and/or chemically cross-linked plurality of bacteriophages with one or more crosslinkers. In some aspects, the plurality of bacteriophages self-assembles into bundles comprising filamentous bacteriophage. In some aspects, the filamentous bacteriophage includes, but is not limited to, the M13 bacteriophage of Escherichia coli.

In some aspects, the hydrogel composition can be used as a therapeutic or diagnostic for biomedical applications, such as tissue engineering therapy, drug delivery, medical device coatings, wound dressings, or biomedical imaging. In some aspects, the hydrogel composition can be used for environmental applications, such as water purification, biomolecule purification, contaminant filtration and removal, or biosensing.

### I. Definitions

Unless otherwise indicated, the definitions and aspects described in this and other sections are intended to be applicable to all aspects of the present invention herein described for which they are suitable as would be understood by a person skilled in the art.

In understanding the scope of the present disclosure, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” (or vice versa) wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effects described herein. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded by way of proviso or negative limitation, such as any specific compounds or method steps, whether implicitly or explicitly defined herein. For example, in aspects, polymers are excluded from the compositions described herein. For example, hydrogel polymers, such as PEG, may in aspects be excluded from the compositions described herein.

In addition, all ranges given herein include the end of the ranges and also any intermediate range points, whether explicitly stated or not.

The term “and/or” as used herein means that the listed items are present, or used, individually or in combination. In effect, this term means that “at least one of” or “one or more” of the listed items is used or present.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. aspect

The term “hydrogel” as used herein refers to a material that exhibits the ability to swell and retain a significant fraction of water within its structure, without dissolving in water. It will be understood that conventional hydrogels typically comprise a water-swellable polymeric matrix, consisting of a three-dimensional network of hydrogel polymers (e.g., hydrophilic polymers, hydrophobic polymers, blends thereof, such as poly(ethylene glycol), collagen, gelatin, dextran, elastin, alginate, hyaluronic acid, poly(vinyl alcohol), derivatives thereof, and combinations thereof) held together by covalent or non-covalent crosslinks. While the hydrogels described herein may comprise such polymers as additional components, the hydrogels described herein may comprise only crosslinked bacteriophages. It has now been found that crosslinked bacteriophages result in a hydrogel composition that can absorb a substantial amount of water (e.g., 50%, 60% 70%, 80%, 90%, 95%, 96%, 97%, 98%, 99% or greater than 99% per unit of non-water molecule) to form a water-swellable gel.

The term “crosslinked” herein refers to a composition containing intramolecular and/or intermolecular crosslinks, whether arising through covalent or noncovalent bonding, and may be direct or include a cross-linker. “Non-covalent” bonding includes both hydrogen bonding and electrostatic (ionic) bonding.

The term “self-healing” as used herein refers to a material that when broken or cut, has the ability to substantially return substantially to an initial state or condition prior to being broken or cut to retain material integrity. This healing process can be aided by stimuli, including but not limited to, electrolytes, ions, proteins and/or peptides, change in temperature and/or pH, or applying an electric or a magnetic field.

### II. Compositions

Described herein is a hydrogel composition comprising cross-linked bacteriophages. In aspects, the hydrogel is a multifunctional hydrogel comprised of self-organized filamentous phage, crosslinked with a crosslinker such as glutaraldehyde, a simple non-responsive crosslinker, which demonstrates three major properties: namely autofluorescence, self-healing, and bioactivity. Highlighted herein is the utility of bacteriophage, such as M13, as a powerful building block for bottom-up assembly of multifunctional bioactive materials with advanced functionalities that make them suitable for theranostics. Paired with the ability of the phage nanofilament to self-replicate and to readily lend itself to genetic engineering, filamentous phage and its self-organized macrostructures are a powerful class of advanced multifunctional bioactive material.

Accordingly, provided herein is a hydrogel composition comprising cross-linked bacteriophages, for example, a physically and/or chemically cross-linked plurality of bacteriophages with one or more crosslinkers. In some aspects, the plurality of bacteriophages self-assembles into bundles. The bacteriophages may be of any type and may infect any bacteria, but in typical aspects, the of bacteriophages comprise filamentous bacteriophages. In some aspects, the bacteriophages comprise Escherichia coli bacteriophages including, but not limited to, f1, M13, or fd bacteriophages, or combinations thereof. Typically, the bacteriophages are M13 bacteriophages.

The bacteriophages may be crosslinked in any manner. Typically, however, chemical or physical crosslinkers are used. In some aspects, the one or more crosslinkers comprises chemical crosslinkers. In some aspects, the crosslinkers are covalent or non-covalent crosslinkers. In some aspects, the covalent or non-covalent crosslinkers comprise bioconjugation reagents including, but not limited to, glutaraldehyde, 1-ethyl-3-(3-dimethylaminopropyl) carbodiimide (EDC), N-hydroxysuccinimide (NHS), epichlorohydrin, biotin, avidin, and streptavidin.

In some aspects, the one or more crosslinkers comprises physical crosslinkers. In some aspects, the crosslinkers include but are not limited to, polyelectrolytes, nanoparticles and/or nanocrystals. In some aspects, polyelectrolytes, nanoparticles and/or nanocrystals include, but are not limited to, poly-L-lysine, metallic nanoparticles, or cellulose nanocrystals.

In some aspects, one or more chemical or physical bonds of the hydrogel degrades under physiological or environmental conditions. In some aspects, the hydrogel is biodegradable.

In some aspects, the hydrogel exhibits self-healing properties. In some aspects, the hydrogel exhibits self-healing properties in the presence of ions, electrolytes and/or proteins. In some aspects, the hydrogel exhibits self-healing properties in the presence of calcium ions and/or phosphate-buffered saline.

In some aspects, the hydrogel is fluorescent. In some aspects, the hydrogel is autofluorescent. In some aspects, the hydrogel is autofluorescent in three channels that decays through biodegradation.

Typically, the hydrogel comprises sufficient bacteriophage to result in one or more of the characteristics described herein. In some aspects, the hydrogel comprises at least about 10⁸ PFU/mL bacteriophage, such as at least about 10⁹ PFU/mL, at least about 10¹⁰ PFU/mL, at least about 10¹¹ PFU/mL, at least about 10¹² PFU/mL, at least about 10¹³ PFU/mL, at least about 10¹⁴ PFU/mL, at least about 10¹⁵ PFU/mL, or at least about 10¹⁶ PFU/mL, such as from about 10⁸ PFU/mL to about 10¹⁶ PFU/mL of bacteriophage. In some aspects, the hydrogel comprises micro- and/or nano-sized particles. In some aspects, the hydrogel is dried to form an aerogel or xerogel. In some aspects, the hydrogel is dried to form an aerogel by critical-point drying or freeze-drying.

In some aspects, the hydrogel is antibacterial. In some aspects, the plurality of bacteriophages of the hydrogel further comprises one or more molecules for cell targeting and/or infectivity. In some aspects, the one or more molecules comprises proteins and peptides. In some aspects, the proteins are antibodies.

In some aspects, the hydrogel is birefringent resulted from the alignment of bacteriophages. In some aspects, the concentration of the hydrogel further changes to achieve different phage alignment, such as nematic phase and smectic phase.

In some aspects, the plurality of bacteriophages of the hydrogel comprises genetically engineered bacteriophage. In some aspects, the plurality of bacteriophages comprises genetically engineered bacteriophage for selective target ligand recognition.

In some aspects, the hydrogel composition further comprises a drug or bioactive agent, wherein the drug or bioactive agent is encapsulated within the hydrogel. For example, one or more antibiotics or other antimicrobial agents may be combined in the hydrogel composition and, optionally, the antibiotic and/or antimicrobial agent may act synergistically with the crosslinked bacteriophages to treat and/or prevent an infection. Other agents may be included in the hydrogel additionally or alternatively, such as hemostatic agents in the case of a wound dressing for example, or a therapeutic agent or angiogenic agent in the case of an implant.

In some aspects, the hydrogel further displays microstructures on the hydrogel surface with the assist of molds to acquire more features. In some aspects, the microbumps and micro suction cups on the hydrogel surface bring adhesive property and large specific surface area.

In some aspects, the hydrogel further transfer to microgels in different shapes with the assist of molds. In some aspects, the microgels can realize most functions of the hydrogel and also provide unique advantages for biosensor and drug delivery.

In some aspects, the bacteriophages within the hydrogel are gene-modified to adjust the length, which make the hydrogel a photonic crystal material with structural colors.

In some aspects, the hydrogel composition further comprises multiple bacteriophage strains which can treat various bacterial infections, especially infections caused by diverse bacteria species. In other aspects, the hydrogel composition comprises multiple bacteriophage strains for treating a single infection.

In some aspects, the hydrogel and microgel composition further comprises polymers to form the hydrogel, wherein the polymers are responsive to environmental stimulations, including but not limited to temperature, pH and light.

Also provided is a therapeutic or diagnostic comprising one or more hydrogel compositions disclosed herein. In some aspects, the therapeutic or diagnostic further comprises a pharmaceutically acceptable carrier, diluent and/or adjuvant. In some aspects, therapeutic or diagnostic comprising the hydrogel composition can be used for biomedical applications including, but not limited to, tissue engineering therapy, drug delivery, medical device coatings, wound dressings, or biomedical imaging.

In another aspect, provided is a scaffold for material synthesis based on the hierarchical and porous structures of the hydrogel. In some aspects, the hydrogel is used to specifically deposit metallic ions or minerals. In some aspects, phage particles are burnt off after the mineralization is complete.

In another aspect, provided is a catalyst with high surface area.

In another aspect, provided is a bioactive membrane or filter comprising one or more of the hydrogel compositions disclosed herein. In some aspects, the bioactive membrane can be used can be used for water purification or other environmental applications including, but not limited to, biomolecule purification or contaminant filtration and removal.

In another aspect, provided is a biosensor substrate comprising one or more of the hydrogel compositions disclosed herein. In some aspect, the biosensor can be used for biomedical and/or environmental applications.

### EXAMPLES

The following non-limiting examples are illustrative of the present invention:

### Example 1. Materials and Methods

Bacteria and phage culture methods: Escherichia coli K12 ER2738 (New England Biolabs Ltd.), genotype: F′proA⁺B⁺ lacI^{q} Δ(lacZ)M15 zzf:Tn10(Tet^{R})/fhuA2 glnV Δ(lac-proAB) thi-1 Δ(hsdS-mcrB)5, was used as host for phage M13. Pre-cultures of host were prepared in LB-Miller broth (Fisher Scientific) using a single colony from fresh tetracycline plates (streaked from glycerol stocks) and incubated shaking at 37° C. overnight. The preculture was then diluted 1:100 in 250 mL of LB broth, to which 10 μL of M13 phage stock at 10¹² plaque forming units (PFU) per mL was added. The phage culture was incubated shaking at 37° C. for 5 hours. The culture was subsequently centrifuged (7000×g, 15 min) to pellet bacteria. The supernatant, containing phage, was saved and stored at 4° C.

Phage purification and concentration: The crude phage stock purified via aqueous two-phase method, as described by Sambrook.¹ Briefly, a mixture of 20 (w/v) % PEG solution and 2.5M NaCl solution was added to the crude phage stock with a volumetric ratio of 1:6 and incubated at 4° C. overnight. Phage was pelleted by centrifugation (5000×g, 45 min, 4° C.). The pellet was resuspended in 10 mL of RO Millipore water (resistivity=18.2 MΩ·cm) and incubated on a tube roller at 4° C. for 2 hrs. Tubes were subsequently centrifuged (5000× g, 15 min) to remove any residual bacterial debris. This purification step was repeated twice and the resulting phage stock was then further purified and concentrated using Amicon Ultra centrifugal filters (Millipore Sigma, Ultra-15, MWCO 100 KDa and 30 KDa sequentially). The concentration of M13 phage was quantified using the plaque assay method, as described elsewhere.²

Preparation of phage hydrogels: Different concentrations of M13 phage suspension were prepared by making serial dilution of the purified, concentrated stock (10¹⁴ PFU/mL) in Millipore water. Next, in a 3 mL disposable syringe, phage was mixed with 1% or 2.5% glutaraldehyde and incubated at room temperature between 12 and 24 hrs. Inversion test was applied to verify the gelation. The hydrogels were then removed from the syringe and kept submerged in Millipore water at room temperature until used for subsequent experiments. Hydrogels were prepared fresh and used within a few hours for all experiments described below. Although they can be stable and stored in DI water at room temperature after producing.

Swelling test: Phage hydrogels were flash frozen with liquid nitrogen to decrease ice crystal formation and immediately freeze dried using Labconco lyophilizer. The dried gels were weighed and placed in 20 mL of 0.1 M pH=7.4 phosphate-buffered saline (PBS) at different temperatures (4, 25, and 37° C.). The swollen gels were removed from the solution at the certain time intervals, after gently removing excess water with lint-free tissue paper, the gels were weighed using a Mettler analytical balance (readability 0.1 mg). Swelling ratio was calculated as follows by using the measure mass of wet gel (m_{w}) and dry gel (m_{d}):

$$swelling ratio ( \% ) = \frac{{m}_{w} - {m}_{d}}{{m}_{d}} \times 100.$$

Compression test and rheometry: Compression tests were carried out at 25° C. using a Mach-1 Mechanical Tester (Biomomentum Inc, QC) with parallel-plate geometry. Prior to mechanical test, phage hydrogel discs were prepared with a diameter of 10 mm and a height of 2-4 mm. Compression testing was performed to 20% of the sample height at a rate of 0.03 mm/s. Preload force of 0.01 N and ramp force of 0.5 N/min were applied.

Rheological properties of runny phage hydrogel (6×10¹² PFU/mL) were determined using an HR-2 Discovery Hybrid Rheometer (TA Instrument) equipped with Peltier plate Steel. A 1.005°, 40 mm cone plate geometry with 100 μm truncation gap was used for all measurements. Dynamic rheological measurements were carried out at 25° C. with a strain sweep between 0.1% and 100% and angular frequency was 10 rad/s.

Chemical characterization and spectrometry: UV-vis spectra of both the phage suspension and phage hydrogels in two different concentrations (3×10¹³ and 3×10¹⁴ PFU/mL) was recorded using a BioTek plate reader in the range of 200-900 nm. Wavelength scanning was performed between 200 nm and 900 nm (whole range of UV-vis spectra) using a 96 well-plate. Attenuated total reflectance infrared (ATR-FTIR) spectra of phage solution and hydrogel were obtained with a Thermo Nicolet IR 560 system, using a Zn—Se ATR accessory (Thermo Electron Corporation, PA). Each sample was placed against the ATR element and the spectra were collected in the range of 500-4000 cm⁻¹ using 128 scans at a resolution of 4 cm⁻¹. After acquisition, the IR spectra were baseline corrected for carbon dioxide peak at approximately 2750 cm⁻¹.

The fluorescence spectra for the phage suspension and the hydrogels was recorded using a Tecan fluorescence spectrometer. Phage hydrogel was prepared in a 3 mL cuvette. Excitation wavelength was set to vary from 370 nm to 600 nm with a 10 nm increments in each scan and emission wavelength was set from a wavelength slightly above the excitation wavelength to 800 nm.

Microscopy: The M13 suspension was coated on a silicon wafer substrate using convective assembly, as described elsewhere.³ The samples were imaged using a Bruker Atomic Force Microscope (AFM) in air under tapping mode using a commercial n+-silicone cantilever, 240 μm long and 35 μm wide, with a resonant frequency of 50-130 kHz and spring constant of 9.0 N/m. The scanning rate was 1.0 Hz, at 0° angle.

For electron microscopy, the samples were prepared using two different methods, namely freeze-drying and critical-point drying. For freeze-drying, hydrogels were flash frozen in liquid nitrogen, freeze dried, then freeze fractured. For critical point drying, the hydrated hydrogels were dehydrated using an ethanol gradient and dried for 3.5 hrs using a Leica critical point dryer (EM CPD300).

The samples were stored in a desiccator and prior to electron microscopy, were coated with a 10 nm layer of gold. Imaging was performed using at 10 kV a field emission scanning electron microscope (TESCAN VP. SEM).

The hydrogels were imaged in hydrated state using a Zeiss inverted fluorescence microscope with three sets of filters, namely ex/em=358/461 nm, ex/em=470/530 nm and ex/em=595/630 nm. The Excitation filter was placed in front of the LED light to excite the hydrogel with the specific wavelength and the Emission filter was attached on the camera to record the emission image of the phage hydrogel.

For polarized microscopy, an enclosed chamber was created using a cleaned and dried glass slide and a cover slip. The coverslip was placed onto the glass slide, secured with parafilm on both sides and heated to 90° C. and removed when the parafilm became transparent. The M13 sample (or Milli-Q water for negative control) was pipetted into the confined chamber, which was then sealed with Vacuum Grease or nail polish, and incubated at 4° C. A saturated NaCl solution (36 (w/v) %) was placed in the petri dish to maintain 75% relative humidity. Samples were examined the following day using a Nikon light microscope equipped with polarizers, which were rotated at different angles using the 10× objective. Images were captured and analyzed using Motic imaging software.

Self-healing: For self-healing experiments, fully hydrated hydrogels were cut into two halves, the halves (from the same or different hydrogels) were put in a same shape mold and kept in contact in the presence of 2.6 mM calcium chloride or phosphate buffered saline (PBS) at room temperature for 24-48 hrs. The healed hydrogel was taken out of the mold and used for either compression test or electron microscopy. Repeated self-healing was performed by cutting a healed hydrogel at a different location and repeating the procedure as described above.

### Example 2. Characterization of Bundles of Nanofilaments

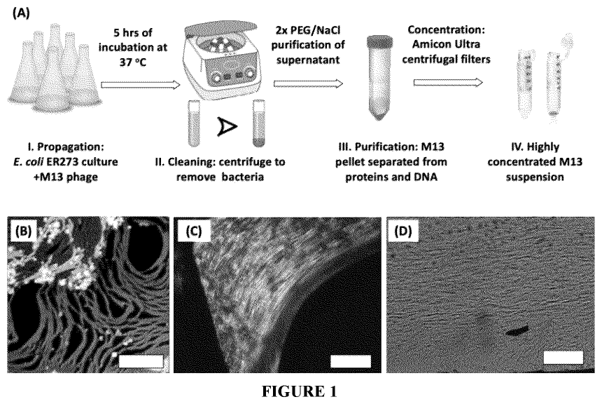

The purified and concentrated phage stock, prepared using the method outlined in FIG. 1A, was quantified to have 6×10¹⁴ PFU/mL. This stock was either used at full strength, or diluted in Millipore water to the desired concentration. A monolayer of M13 phage stock, assembled on silicon wafer can be seen in FIG. 1B. Each phage filament is 6-8 nm thick and 850 nm long. Therefore, the filaments visible in FIG. 1B are bundles of phage and not a single filament.

Self-organization was induced in M13 suspensions with concentrations higher than 10¹⁴ PFU/mL via confinement at low temperature. The polarized light micrograph of the aligned M13 is presented in FIG. 1C, compared to the micrograph recorded without cross-polarizers (FIG. 1D). M13 has been reported to self-organize into lyotropic liquid crystals at 14-22 mg/mL (nematic phase) and 28-76 mg/mL (chiral nematic phase).⁴ That is equivalent to 1×10¹⁴-1×10¹⁵ phage particles per mL. It is noteworthy that the concentration of M13 was determined using the standard plaque assay, which estimates the number of virions based on the number of virions that can form a plaque on a bacterial lawn. This method is the gold standard in microbiology but is known to underestimate the number of virions; therefore, the actual number of virions in the M13 suspension may be up to ten times higher than that determined through the plaque assay.

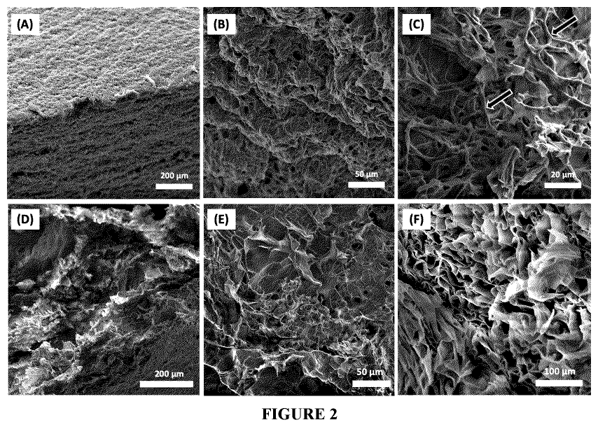

SEM images of the hydrogels (crosslinked with 1(w/v) % glutaraldehyde, critical-point dried) confirmed a highly ordered structure. FIG. 2A shows long-range order to the hydrogel layers. At higher magnification, a porous structure was observed (FIG. 2B), composed of filaments, the size of which suggests M13 filament bundles of several hundred phages (FIG. 2C). Decreasing the phage concentration 10-fold (to 3×10¹³ PFU/mL) appears to decrease the long-range order of the hydrogel microstructure (FIG. 2D), however a level of order still prevailed at higher magnification and filaments with a size suggestive of phage bundles of several hundred were visible (FIG. 2E). For comparison, FIG. 2F, shows the structure of a freeze-dried hydrogel, which, as expected, shows a different pore structure than critical-point dried hydrogels, likely as a results of ice crystal formation. The ice crystals appear to have molded the phage bundles into thin sheets of M13, enveloping the larger pores, formed around the crystals.

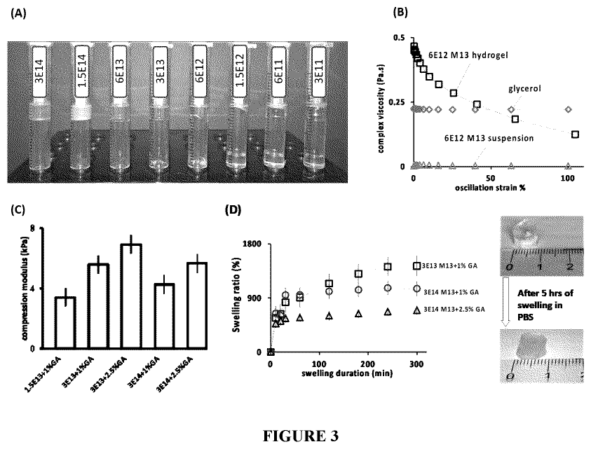

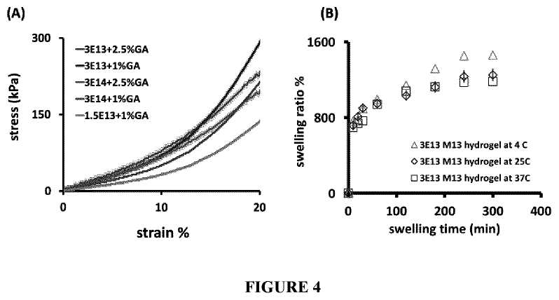

M13 phage formed hydrogels with concentrations as low as 6×10¹² PFU/mL (FIG. 3A). The test tube inversion method was used to confirm gelation. Depending on M13 phage titer, gelation time varied between 12 and 24 hrs. The gels formed with 6×10¹² PFU/mL were very soft and did not hold their shape. When compared to the viscosity of a viscous liquid (glycerol) and a non-crosslinked phage suspension, the liquid-like hydrogel formed with 6×10¹² PFU/mL of M13 showed a viscosity representative of crosslinked polymers and distinctly different from viscous liquids and the phage suspension as non-crosslinked liquid (FIG. 3B). For all subsequent experiments, only hydrogels prepared with higher phage concentrations (higher than 6×10¹³ PFU/mL) were used. The stress-strain curve is presented in FIG. 4A, which shows a typical trend for protein hydrogels with no creep region. Moreover, the experimental observation showed that there was no breaking point of the hydrogels under pressure up to 25 N, although they were pressed with dimensional changes under this force. The M13 hydrogels exhibited a compression modulus of 5-9 KPa. The nonlinear nature of the stress-strain curve complicates the calculation of a global modulus of compression, therefore the slope of the stress-strain curve at 0-5% strain was calculated. Compression modulus increased slightly with increasing phage concentration; however, increasing the crosslinking density (using 2.5% glutaraldehyde rather than 1%) appeared to have a more marked effect on the compression strength of the hydrogels (FIG. 3C). The crosslinking reaction occurs between ϵ-amino groups of lysine residues of the major capsid protein of M13 phage and two aldehyde groups in glutaraldehyde. During the process of gelation, glutaraldehyde, an aggressive carbonyl (—CHO) reagent, condenses amine groups of lysine residues on the phage coat protein, around 340 available lysine residues,⁵ via Mannich reaction or reductive amination. Higher glutaraldehyde concentration suggests a higher crosslinking density, creating more junction points along each filament. This in turn can increase the stiffness of the hydrogel network, as demonstrated in FIG. 3C.

The increase in network/crosslinking density, however, has an inverse effect on the M13 hydrogel swelling ratio. The hydrogel prepared with 3×10¹³ PFU/mL of M13 possesses the highest ratio of swelling of 16 times their dry weight (FIG. 3D). The hydrogel prepared with 3×10¹⁴ PFU/mL of M13 only absorbed 11 times their dry weight in water. When the crosslinking density was increased (using 2.5% glutaraldehyde 3×10¹⁴ PFU/mL) the swelling ratio was only 700%. No temperature effect was observed for swelling (FIG. 4B).

### Example 3. Biodegradation-Dependent Fluorescence of Hydrogels

During the gelation process, a change in color was observed where the colorless, clear M13 phage suspension turned to a hydrogel with a yellow hue, more visible at higher M13 concentrations, which can be due to cyclic hemiacetal formations with glutaraldehyde. A UV-vis spectrum showed that M13 phage suspension has a sharp absorption peak at λ=280 nm, a typical absorption peak for proteins (FIG. 5A). UV-Vis spectrum scanning from 200 nm to 500 nm for the M13 phage hydrogel demonstrated a new, relatively broad peak around 310 nm. To further investigate chemical change in the hydrogel, FTIR spectra of the hydrogel was analysed in comparison to the M13 suspension. Both the phage suspension and the hydrogel showed the same amide I peaks at 1660 cm⁻¹, demonstrating amide bond in protein capsid of M13 phage (FIG. 5B). In addition, the hydrogel exhibited amide II (˜1570 cm⁻¹) and amide III bonds (1280 cm⁻¹), marked on FIG. 5B. There are multiple new absorption peaks (aside from those shared with glutaraldehyde and M13 suspension) that suggest new functional groups/new compounds formed. During the crosslinking reaction, α,β-unsaturated aldehyde polymer can be formed, which leads to the C═C double bond existed in the system, accompanied by the C═N double bonds from Schiff's base. The reaction between glutaraldehyde and amine groups may lead to side products, including Schiff's base with C═N bond, α,β-unsaturated (conjugated) Schiff's base with C═C bond, Michael addition product formed from α,β-unsaturated products, and dihyropyridine or dihydropyridinium which follow the ring closure of an intermediate during the reaction. FT-IR peaks on FIG. 5B shows a peak around 1480 cm⁻¹ corresponding to imine (C═N) bond, and peaks between 1000 and 1200 cm⁻¹ indicating C—N bond formed with Michael addition.

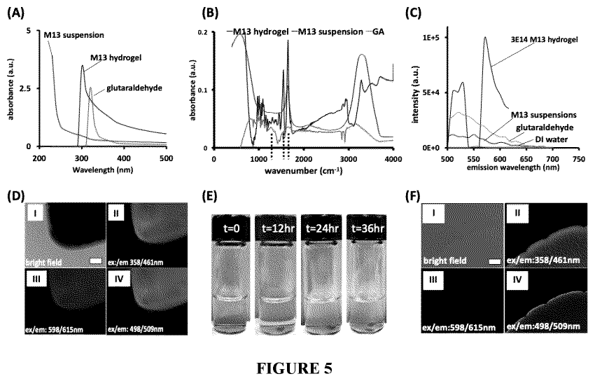

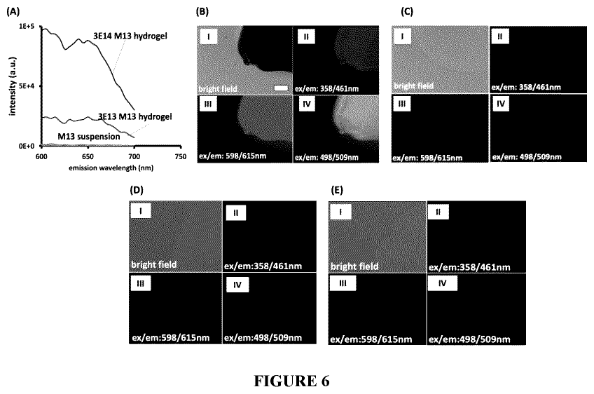

Further spectrometry revealed the M13 hydrogels to exhibit autofluorescence when excited at specific wavelengths. Emission spectra of M13 hydrogels and M13 suspensions were monitored with a fluorometer and the results show distinct peaks when excited at 2=470 nm (FIG. 5C), and 2=598 nm (FIG. 6A). As shown in FIG. 5C, the spectra for glutaraldehyde, water, and M13 phage suspension do not show any similar peak on the same wavelengths. Autofluorescence was confirmed using microscopy with three different optical filter sets (ex/em=358/461 nm, ex/em=498/509 nm, ex/em=598/615 nm) and the hydrogels show blue, green and red autofluorescence (FIG. 5D for 3×10¹⁴ PFU/mL, FIG. 6B for 3×10¹³ PFU/mL,). Neither M13 phage suspensions (FIG. 6C, D) nor glutaraldehyde (FIG. 6E) show any autofluorescence when imaged with the same set of filters. Therefore, both spectrometry and microscopy suggest the existence of fluorescent molecule/functional group produced by M13 crosslinking with glutaraldehyde. However, this functional group could not be resolved via FTIR. The fluorescence observed in the M13 hydrogel may be attributed, at least in part, to the electronic transitions such as π-π* transition of C═C bond in glutaraldehyde and n-π* transition of C═N bonds in the Schiff's base (result of reaction between α,β-unsaturated aldehyde polymers in glutaraldehyde and M13 surface proteins). Glutaraldehyde reacts with proteins and peptides to generate visible to near-IR emitters such as ethylenediamine and secondary amines. The nature of this autofluorescence is believed to be dependent on the type of protein that reacts with glutaraldehyde, size of the protein or peptide and pH of the reaction, thus offering a versatile platform for preparing hydrogels for imaging applications.

Furthermore, when the M13 phage hydrogels were degraded using a 1 mg/mL solution of proteinase K (FIG. 5E), the degradation products also exhibited autofluorescence, but not in the red channel (FIG. 5F). Proteinase K has the ability to hydrolyze the amine bonds and break the ester bonds process in order to track the color changes. As shown in FIG. 5E, the M13 phage hydrogel was mostly digested after 36 hrs at 37° C. The color of the solution changed to yellow over time, consistent with the color of the hydrogel, which suggests the release of the fluorescent molecules produced in the crosslinking process, which was confirmed via fluorescence microscopy.

It is noteworthy that the red autofluorescence (ex/em 598/618) was no longer observed for the degradation product, suggesting that the biodegradation could be remotely tracked for scenarios where the hydrogel is used for tissue engineering applications. Autofluorescence in hydrogels has been previously reported for BSA hydrogels,⁵ among others and shown to be attractive for non-invasive tracking of hydrogels in the body.⁶ One advantage of the M13 hydrogels over the other hydrogels reported in the literature is that the degradation products can also be tracked in other channels while degradation is tracked in the red channel.

### Example 4. Self-Healing and Bioactivity of Phage Hydrogels

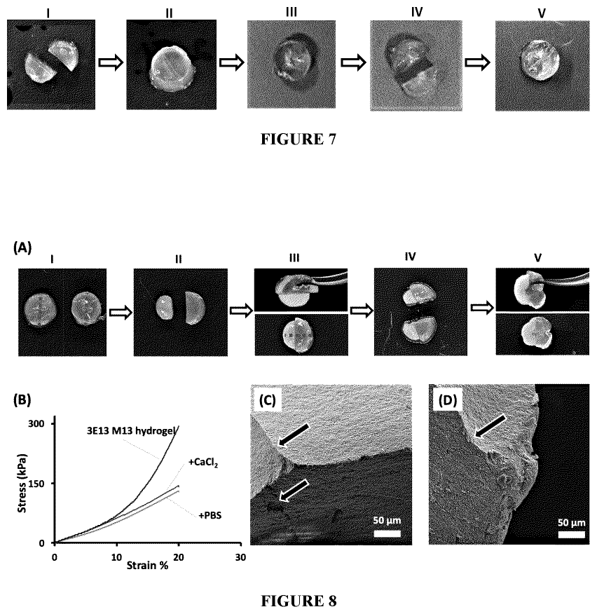

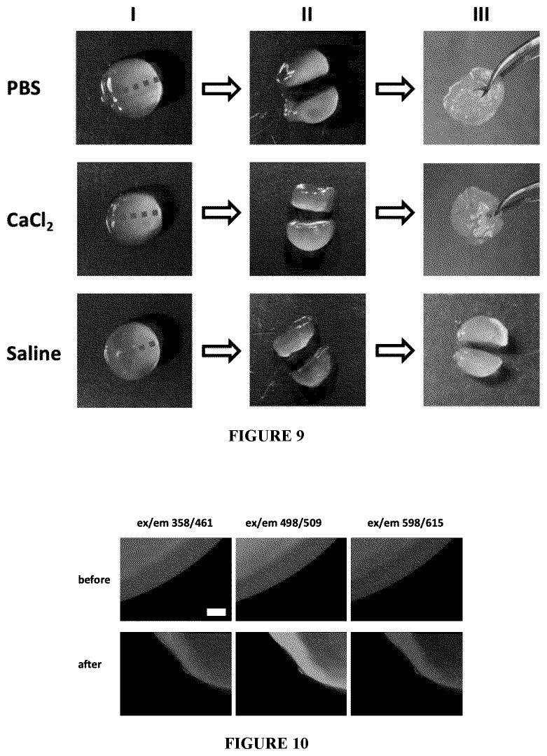

The M13 hydrogels prepared with both ˜10″ and ˜10¹³ PFU/mL of M13 exhibited repetitive self-healing capability at room temperature (FIG. 7). Furthermore, M13 phage hydrogels made with two different concentrations of M13 suspension were shown to fuse together when put in contact in the presence of Ca²⁺ (FIG. 8A) or PBS (FIG. 9). When the hydrogels, cut with a razor blade, were incubated at room temperature in the presence of 2.6 mM CaCl₂ (extracellular physiological levels) or PBS, the hydrogels started to show signs of healing within hours, completely fusing back together into an intact hydrogel in 24 hrs (for CaCl₂) and 47 hrs (for PBS). The fused hydrogel showed a lower compression modulus than the intact hydrogels (˜4.4 and ˜6.1 compared to ˜9 for an intact hydrogel made with 3×10¹³ PFU/mL of M13), suggesting a degree of compromised mechanical strength (FIG. 8B). Electron microscopy of the fused gel showed a scar than ran across the body of the gel, causing a disconnect in the order of the layers, but the layers were nonetheless fused together at the microscale (FIG. 8C,D).

The M13 hydrogels that were incubated in the presence of Ca²⁺ or PBS exhibited a similar stress-strain response (FIG. 8B). This observation suggests that residual aldehydes may be reacting with the amines in or re-reacting with new lysins on M13. Since the crosslinking is probably due to Schiff's base, self-healing may be the reformation of Schiff's bases, most likely assisted by M13/calcium interactions.

There are no reports of M13 being physically crosslinked with Ca²⁺ at room temperature, however BSA has been reported to be physically crosslinked with Ca^{t} after being first heated to thermally unfold the native BSA protein and form fibrillar BSA aggregates, then gelation is induced by adding fresh native BSA into the solution in the presence of Ca²⁺ at room temperature.⁷ In summary, the observation that the M13 hydrogels heal under physiological calcium concentration suggests that these hydrogels can be expected to exhibit self-healing similar to biological tissue where the healing processes could be autonomously triggered after the damage. FIG. 10 shows that hydrogel autofluorescence is preserved after self-healing, namely after being incubated in PBS or CaCl₂ for 24 hrs.

The biological activity of the M13 phage hydrogels was confirmed by probing the ability of the constituting phage fibers to infect their bacterial host. The hydrogels were incubated with E. coli ER2738 (5 hrs, 37° C.), the culture showed a relatively high titer of phage suggesting the M13 phage inside the hydrogel, even though fixed in a matrix and chemically crosslinked, was still biologically active and able to infect its host bacteria and propagate itself (FIG. 11A). To rule out the possibility of loosely bound M13 leaking out of the hydrogels, phage infectivity for freshly prepared hydrogels was compared with hydrogels that were washed in Millipore water (10 water changes, 25 mL each). The washed hydrogel resulted in the same level of propagated phage as the non-washed hydrogel, when incubated with host bacteria (FIG. 11A). This result also indicates that the any possible residues of the glutaraldehyde crosslinker used to prepare the hydrogel, was below toxicity levels to bacteria, because the bacteria were physiologically active to propagate the phage. The phage count in the wash waters used to wash the hydrogels was further quantified. The concentration of M13 phage in the wash waters was below detection limit of the standard plaque assay, indicating minimal leaking out of M13 phage out of the hydrogels. In addition, after incubating a fresh hydrogel in wash buffer for 5 hrs, the level of phage in the wash buffer was below detection limit of a standard plaque assay. This evidence further confirms that M13 propagation in the presence of hydrogels in not primarily a result of leaking of free M13 phage, but mainly a result of the preserved biological activity of M13 in the hydrogels. For M13 phage to infect its host bacteria, the phage uses a protein known as g3p (protein encoded by gene 3), a ˜50 KDa protein situated protruding from the tip of the M13 phage filament with a copy number of 5 (FIG. 11B). In fact, the lighter spots (higher elevation) in the AFM image in FIG. 1B are the protruding g3p's. It is proposed that the g3p's for some of the M13 filaments, crosslinked in the hydrogel network are exposed and available through the surface of the hydrogel and the surface of the pores to bind host bacteria (FIG. 11B). Binding is known to initiate the insertion of the M13 genome into the bacterial cell and start the infection and M13 propagation cycle.⁸ This means that even if M13 filaments are not available to freely diffuse through the environment and bind to their host cells (as they do in the typical phage propagation cycle), they can still infect host bacteria, thus maintaining their cherished biological activity in the hydrogel. The main bottleneck in achieving bioactive phage hydrogels was adjusting the ratio of crosslinker to M13 so as to avoid over crosslinking and self-crosslinking of the phage proteins. The detection of progeny phage in the bioactivity experiment confirms that it is possible to design the crosslinking reaction such that the surface proteins remain biologically active. As g3P is the protein most widely used for phage display, a screening technology aimed at uncovering peptides that show high specificity for organic and inorganic substrates of interest, M13 hydrogels that show specific affinity towards targets as diverse as environmental contaminants and cancer cell biomarkers may be developed. The bioactivity of the M13 hydrogels is, however, not limited to g3p; protein 8 (coating the entire length of the fiber, minus the two ends) is also widely used for expressing recombinant peptides. This protein has a significantly higher copy number of 2500-2700 and coats the entire phage filament (except for the tips), thus offering more binding sites for binding of desired ligands in a M13 hydrogel made with genetically engineered M13 filaments, further expanding the applications and impact of M13 hydrogels as a multifunctional material.

### Example 5. Birefringence of Phage Hydrogels

Birefringence is an optical property of a material having a refractive index that depends on the polarization and propagation direction of light and can be seen under a polarized light microscope. It is defined as the maximum difference between the minimum and maximum indexes of refraction. The phenomenon can be illustrated as follows: when inbound light, which is made up of wavelengths of varying directions and angles, interacts with a polarizer in front of a material, only light parallel to that polarizer will be allowed to pass through and interact with that material. If the material is birefringent, then the inbound light will be rotated 90 degrees such that it will be allowed to pass through a second polarizer that is perpendicular to the first and placed after the material. If the sample is not birefringent, then the light won't be rotated 90 degrees, nor will it be able to pass through the second polarizer.

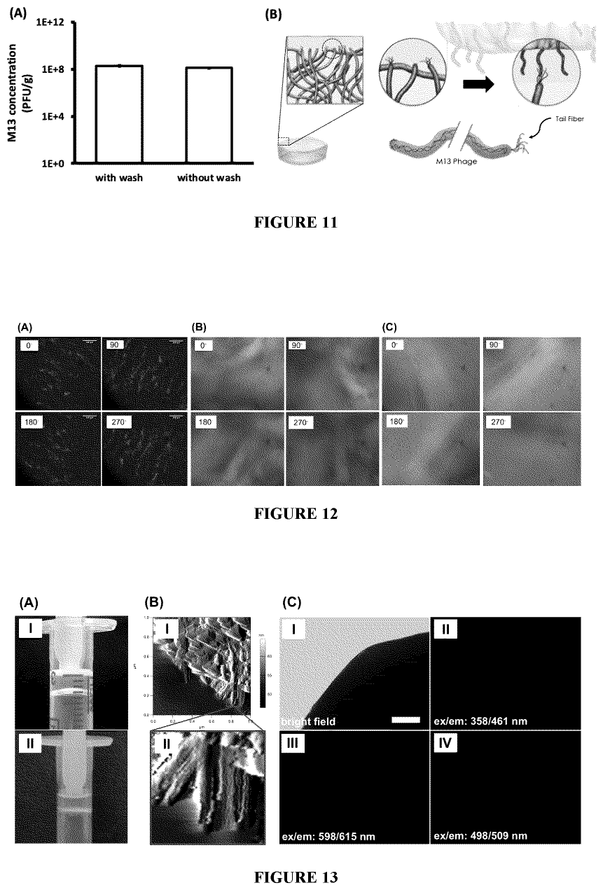

When M13 bacteriophages self-assemble, they are able to display birefringent properties due to their inherent shape, length, and molecular properties. In FIG. 12A, it is shown that when M13 bacteriophages are present in solution at high concentration, they are able to naturally self-assemble and display birefringent properties when rotated 360° on a polarized light microscope. FIG. 12B and FIG. 12C depict two separate M13 bacteriophage-based hydrogels still able to retain their characteristic birefringent properties when rotated 360° on a polarized light microscope. It is therefore demonstrated that even with the addition of a crosslinking agent, in the form of glutaraldehyde at a 1% solution concentration, to form a hydrogel, the M13 bacteriophage are still able to retain their characteristic self-assembly tendencies and their ability to display birefringent properties.

### Example 6. Phage Hydrogels with Different Crosslinkers

Crosslinkers with different mechanism could bring the produced hydrogel different properties. The resulted changes of infectivity, degradation rate, hydrophobicity, swelling ratio and porosity could eventually affect their applications on antimicrobial and drug release. The feasibility of using EDC as the crosslinker was confirmed after observing gelation of 5×10¹³ PFU/mL M13 phage hydrogels with different concentration of EDC (FIG. 13A). Meanwhile, the M13 phages inside the hydrogel showed ordered alignment (FIG. 13B). Comparing to the M13 phage hydrogel crosslinked by glutaraldehyde, the EDC hydrogel did not exhibit autofluorescence (FIG. 13C).

Crosslinker for phage gelation is not limited to glutaraldehyde and EDC. Other chemical crosslinkers and physical crosslinkers, such as NHS, epichlorohydrin, biotin, avidin, streptavidin and gold nanoparticles are expected to have similar gelling ability.

### Example 7. Phage Hydrogels with Microstructures and Microgels

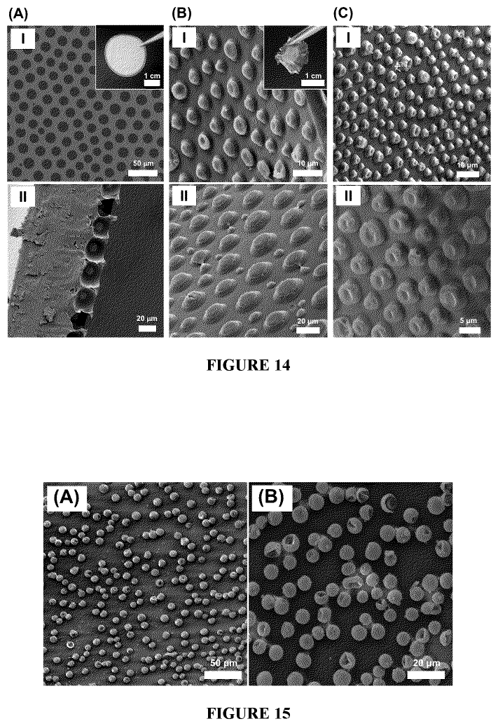

There is no report on designing microstructures on phage hydrogel. This proposed method used porous films as molds to fabricate microdot array on the hydrogel surface. The microdot array can provide a higher specific surface area comparing to flat hydrogel surface. The size and shape of the microdots may be dependent on the microstructures of molds. For example, a mold with spherical pores (FIG. 14A) can create phage hydrogel displaying ordered globular microbumps on the surface (FIG. 14B).

Furthermore, microscale suction cups were formed after putting hydrogel with microbumps inside ethanol. The rapid dehydration of hydrogel resulted in the collapse of microbumps, forming microscale suction cups (FIG. 14C). This structure was found irreversible and has potential to fabricate glue-free adhesive antimicrobial or biosensing patch.

Meanwhile, microgels were fabricated also using porous films as molds (FIG. 15). The specialty and novelty of those phage microgels include two main aspects: firstly, there is no report on making phage microgels. Comparing to phage hydrogel films or bulks, microgels are expected to exhibit advantages in the fields of biosensors and drug delivery. Secondly, the size and shape of the obtained microgels can be controlled using different molds or simply adjusting the molds. For example, spindle pore with different aspect ratios can be obtained through stretching the templates with spherical pores to different elongation, which leads to spindle microgels. These sharp-edge microgels are expected to break into biofilm and exist longer in vivo.

While the present invention has been described with reference to examples, it is to be understood that the scope of the claims should not be limited by the aspects set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in herein is found to be defined differently in a document incorporated herein by reference, the definition provided herein is to serve as the definition for the term.

### REFERENCES

1. Sambrook, J.; Russell, D. W., Molecular cloning: a laboratory manual. 3 ed.; Cold Spring Harbour laboratory Press: Cold Spring Harbour, N Y, 2001.
2. Kropinski, A. M.; Mazzocco, A.; Waddell, T. E.; Lingohr, E.; Johnson, R. P., Enumeration of Bacteriophages by Double Agar Overlay Plaque Assay. In Bacteriophages: Methods and Protocols, Volume 1: Isolation, Characterization, and Interactions, Clokie, M. R. J.; Kropinski, A. M., Eds. Humana Press: Totowa, N.J., 2009; pp 69-76.
3. Malaquin, L.; Kraus, T.; Schmid, H.; Delamarche, E.; Wolf, H., Controlled Particle Placement through Convective and Capillary Assembly. Langmuir 2007, 23, (23), 11513-11521.
4. Lee, S.-W.; Mao, C.; Flynn, C. E.; Belcher, A. M., Ordering of Quantum Dots Using Genetically Engineered Viruses. Science 2002, 296, (5569), 892-895.
5. Ma, X.; Sun, X.; Hargrove, D.; Chen, J.; Song, D.; Dong, Q.; Lu, X.; Fan, T.-H.; Fu, Y.; Lei, Y., A Biocompatible and Biodegradable Protein Hydrogel with Green and Red Autofluorescence: Preparation, Characterization and In Vivo Biodegradation Tracking and Modeling. Sci. Rep. 2016, 6, 19370.
6. Fei, J.; Zhao, J.; Du, C.; Wang, A.; Zhang, H.; Dai, L.; Li, J., One-Pot Ultrafast Self-Assembly of Autofluorescent Polyphenol-Based Core@Shell Nanostructures and Their Selective Antibacterial Applications. ACS Nano 2014, 8, (8), 8529-8536.
7. Chen, J.; Dong, Q.; Ma, X.; Fan, T.-H.; Lei, Y., Repetitive Biomimetic Self-healing of Ca²⁺-Induced Nanocomposite Protein Hydrogels. Sci. Rep. 2016, 6, 30804.
8. Calendar, R. L., The Bacteriophages. second ed.; Oxford University Press: New York, 2006.

1. A hydrogel composition comprising cross-linked bacteriophages.

2. The hydrogel composition of claim 1, wherein the bacteriophages self-assemble into bundles.

3. The hydrogel composition of claim 1, wherein the bacteriophages comprise filamentous bacteriophages.

4. The hydrogel composition of claim 1, wherein the bacteriophages comprise Escherichia coli bacteriophages.

5. The hydrogel composition of claim 1, wherein the bacteriophages comprise f1, M13, or fd bacteriophages, or combinations thereof.

6. The hydrogel composition of claim 1, wherein the bacteriophages comprise M13 bacteriophages.

7. The hydrogel composition of claim 1, wherein the bacteriophages comprise covalent crosslinks.

8. The hydrogel composition of claim 1, wherein the bacteriophages comprise non-covalent crosslinks.

9. The hydrogel composition of claim 1, wherein the bacteriophages are crosslinked with one or more crosslinkers comprising polyelectrolytes, nanoparticles and/or nanocrystals.

10. The hydrogel composition of claim 9, wherein the one or more crosslinkers comprises glutaraldehyde.

11. The hydrogel composition of claim 1, wherein the hydrogel exhibits one or more of the following properties:
bioactivity, for example antibacterial activity;
degradability, such as biodegradability;
self-healing, optionally in the presence of calcium ions and/or phosphate-buffered saline;
fluorescent, such as autofluorescent; and/or
birefringence.

12. The hydrogel composition of claim 1, wherein the hydrogel comprises at least about 10⁸ PFU/mL bacteriophage, such as at least about 10⁹ PFU/mL, about 10¹⁰ PFU/mL, about 10¹¹ PFU/mL, about 10¹² PFU/mL, about 10¹³ PFU/mL, about 10¹⁴ PFU/mL, about 10¹⁵ PFU/mL, or about 10¹⁶ PFU/mL, such as from about 10⁸ PFU/mL to about 10¹⁶ PFU/mL of bacteriophage.

13. The hydrogel composition of claim 1, wherein the hydrogel is dried to form an aerogel or xerogel.

14. The hydrogel composition of claim 13, wherein the hydrogel is dried to form an aerogel by critical-point drying or freeze-drying.

15. The hydrogel composition of claim 1, further comprising one or more molecules for cell targeting and/or infectivity.

16. The hydrogel composition of claim 1, comprising genetically engineered bacteriophages.

17. The hydrogel composition of claim 16, wherein the bacteriophages are genetically engineered for selective target ligand recognition.

18. The hydrogel composition of claim 1, wherein the length of the filamentous bacteriophages is further tuned through gene-modification, giving the phage-composed hydrogel structure colors.

19. The hydrogel composition of claim 1, comprising at least two different bacteriophage strains.

20. The hydrogel composition of claim 19, wherein the at least two different bacteriophage strains target the same bacterial species or different bacterial species to treat complex infections.

21. The hydrogel composition of claim 1, further comprising at least one polymer.

22. The hydrogel composition of claim 21, wherein the at least one polymer comprises PEG.

23. The hydrogel composition of claim 1, excluding a polymer, for example excluding PEG.

24. The hydrogel composition of claim 1, further comprising microstructures on the surface.

25. The hydrogel composition of claim 1, further for transferring to microgels.

26. The hydrogel composition of claim 1, further comprising a bioactive agent, optionally wherein the bioactive agent is encapsulated within the hydrogel.

27. The hydrogel composition of claim 26, wherein the bioactive agent comprises an antibiotic, optionally wherein the bacteriophages and antibiotic are in synergistic amounts.

28. A therapeutic or diagnostic comprising the hydrogel composition of claim 1.

29. The therapeutic or diagnostic of claim 28 further comprising a pharmaceutically acceptable carrier, diluent and/or adjuvant.

30. The therapeutic or diagnostic of claim 28, wherein the therapeutic or diagnostic comprises a wound dressing

31. A scaffold for material synthesis comprising one or more of the hydrogel compositions of claim 1.

32. A catalyst comprising one or more of the hydrogel compositions of claim 1.

33. A bioactive membrane or filter comprising one or more of the hydrogel compositions of claim 1.

34. A biosensor substrate comprising one or more of the hydrogel compositions of claim 1.

35. A method for making a hydrogel composition, the method comprising crosslinking a suspension of bacteriophages.

36. The method of claim 35, wherein the bacteriophages are in a water suspension.

37. The method of claim 35 wherein the suspension comprises at least about 10⁸ PFU/mL bacteriophage, such as at least about 10⁹ PFU/mL, about 10¹⁰ PFU/mL, about 10¹¹ PFU/mL, about 10¹² PFU/mL, about 10¹³ PFU/mL, about 10¹⁴ PFU/mL, about 10¹⁵ PFU/mL, or about 10¹⁶ PFU/mL, such as from about 10⁸ PFU/mL to about 10¹⁶ PFU/mL of bacteriophage.

38. The method of claim 35, wherein crosslinking comprises mixing the bacteriophages with a crosslinking agent.

39. The method of claim 38, wherein the cross-linking agent comprises glutaraldehyde, optionally at from about 0.1% to about 10%, such as from about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, or about 9.5% to about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5% or about 10%.

40. The method of claim 38, wherein mixing comprises incubating with the crosslinking agent for a period of time, such as from about 1 hour to about 48 hours, such as from about 12 hours to about 24 hours, at a temperature of from about 4° C. to about 37° C., such as about room temperature.

41. A hydrogel composition made by the method of claim 35.
